(19) United States
(12) Patent Application Publication
(10) Pub. No.: US 2022/0347211 A1
(43) Pub. Date: Nov. 3, 2022

(54) PROCESS FOR PREPARATION OF WATER HAVING THERAPEUTIC BENEFITS USING SHUNGITE

(71) Applicant: Koteshwar Rao VAKKANTI, Nalgonda, Telangana (IN)

(72) Inventor: Koteshwar Rao VAKKANTI, Nalgonda, Telangana (IN)

(21) Appl. No.: 17/267,600

(22) PCT Filed: Aug. 10, 2019
(86) PCT No.: PCT/IB2019/056820

§ 371 Date: Feb. 10, 2021

(30) Foreign Application Priority Data
Aug. 10, 2018 (IN) 201841030230

(51) Int. Cl. A61K 35/08; A61P 31/04; A61P 31/14; B01J 20/20; B01J 20/28; B01J 20/30; C02F 1/28

(52) U.S. Cl. CPC A61K 35/08; A61P 31/04; A61P 31/14; B01J 20/20; B01J 20/28016; B01J 20/28004; B01J 20/3021; B01J 20/3071; C02F 1/281; C02F 2303/04; C02F 2103/026

(57) ABSTRACT

The present invention generally relates to purifying and processing water using mineraloid material. Specifically, the present invention relates to a process for purification or treatment of water shungite stone and the purified water possess therapeutic effects. The purified shungite treated water of the present invention is useful in the treatment, control or prevention of cancer, inflammatory diseases, viral infection, bacterial infection cardiovascular diseases or gastrointestinal diseases.

### FIELD OF THE INVENTION

The present invention generally relates to purifying and processing water using mineraloid material. Specifically, the present invention relates to process for purification or treatment of water using shungite stone and the purified or treated water possess therapeutic benefits such as anti-inflammatory activity, antiviral activity, antibacterial activity, antimicrobial activity, anticancer activity or cardiovascular effects.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Water is of fundamental source of all life including human life and availability of fresh water, the nature's gift controls the major part of the world economy. Human body contains 70% water, clean and healthy drinking water, determine the human health and longevity.

Drinking water contamination is a major health concern all over the world, especially in the developing and under-developed countries. Various contaminants affect the quality of drinking water, which include microbial contaminants (for example, pathogens like bacteria, viruses, and parasites such as microscopic protozoa and worms), inorganic contaminants (for example, fluoride, arsenic, iron, magnesium, and nitrate), organic contaminants (for example, pesticides and volatile organics) and radiological contaminants.

A number of governmental agencies are working together to address the drinking water contamination problem. Despite all the efforts made so far, contradicting reports continue to emerge on the availability of protected drinking water. Further, a significant cost burden still remains on the shoulders of the poor on treatment of waterborne diseases. Therefore, there remains an unmet need for protected drinking water especially having therapeutic benefits such as anti-inflammatory activity, antiviral activity, antibacterial activity, anticancer activity or cardiovascular effects. Further, providing purified water with therapeutic benefits will reduce the cost involved in the treatment of various diseases.

Shungite, a natural composite, is an excellent and almost universal natural adsorbent which holds on its surface more than 94% of all kinds of harmful impurities contained in tap water. The study of molecular composition of shungite reveals the existence of a special form of carbon-fullerene, which is a hollow spherical molecular compound containing 60, 70 and 84 carbon atoms. It is the presence of fullerenes in the composition of shungite and the unusual structure of the carbon matrix is a characteristic feature of the structure of this mineral, which determines a number of its unique physical, chemical and therapeutic properties. Shungite stone ability to actively absorb oxygen has numerous practical confirmations, and to interact with it actively at indoor temperatures in water and in the air and are useful in the purification of water.

Different water purification and treatment techniques are available in the art. However, these techniques possess drawbacks like cumbersome process, high cost, requires sophisticated apparatus etc.,

Therefore, there is a need for water purification and processing material which solve the existing problems, and having anti-inflammatory activity, antiviral activity, antibacterial activity, anticancer activity or cardiovascular effects. Advantageously, the present invention uses shungite stone for purification and processing of water which is ease of use, low cost, and provides purified shungite treated water having anti-inflammatory activity, antiviral activity, antibacterial activity, anticancer activity or cardiovascular effects.

### OBJECTS OF THE INVENTION

An object of the present invention is to provide a process for purification or treatment of water to obtain purified treated water having anti-inflammatory activity, antiviral activity, antibacterial activity, anticancer activity or cardiovascular effects.

Another object of the present invention is to provide a cost effective, easy to use process of purification or treatment of water using shungite stone.

Another object of the present invention is to provide a process of purification or treatment of water fulfilling the WHO standards of drinking water and having anti-inflammatory activity, antiviral activity, antibacterial activity, anticancer activity or cardiovascular effects.

### SUMMARY OF THE INVENTION

In one aspect, the present invention provides a process for purification and treatment of water using shungite stone.

In another aspect, the process for purification or treatment of water comprising the steps of:
(a) crushing shungite stone into 20 mm to 40 mm pieces;
(b) washing the shungite stone pieces using water;
(c) adding the washed shungite stone pieces into the stainless steel tank;
(d) filling the tank with water and stirring the water and the washed shungite stone pieces for 5 to 10 minutes at an interval of 8 hours for 45 to 50 hours; and
(e) filtering the water obtained in step (d) to obtain purified shungite treated water.

In another aspect of the present invention, the water in the process for purification or treatment is tap water i.e., raw water.

In another aspect of the present invention, the water in the process for purification or treatment is reverse osmosis water.

In another aspect of the present invention, the water in the process for purification or treatment is reverse osmosis (RO), ultra violet (UV) and ozonation processed water.

In yet another aspect, the present invention provides a process for purification or treatment of water using shungite, wherein the amount of shungite stone for 100 litres of water is in the range of 10 to 15 kg.

In yet another aspect, the present invention provides a process for purification or treatment of water using shungite, wherein the amount of shungite stone for 100 litres of water is 12.5 kg.

In yet another aspect, the present invention provides a process for purification or treatment of water using shungite, wherein the amount of shungite stone for 100 litres of water is 10 kg.

In yet another aspect, the present invention provides a process for purification or treatment of water using shungite, wherein the amount of shungite stone for 100 litres of water is 15 kg.

In yet another aspect, the present invention provides a process for purification or treatment of water using shungite, wherein the purified shungite treated water fulfils the requirement of WHO standards.

In yet another aspect, the present invention provides a process for purification or treatment of water using shungite, wherein the purified shungite treated water is free of yeast and moulds, Pseudomonas aeruginosa, Escherichia coli, Coliform, Staphylococcus aureus, Faecal Streptococci, Shigella, Salmonella, Vibrio cholera, Vibrio parahaemolyticus and Sulphite Reducing Anaerobes.

In yet another aspect, the present invention provides a process for purification or treatment of water using shungite, wherein the purified shungite treated water possesses anti-inflammatory activity, antiviral activity, antibacterial activity, anticancer activity or cardiovascular effects.

Various objects, features, aspects and advantages of the inventive subject matter will become more apparent from the following detailed description of preferred embodiments

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Exemplary embodiments will now be described more fully hereinafter in which exemplary embodiments are shown. This disclosure may however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

All processes described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

It should also be appreciated that the present invention can be implemented in numerous ways, including as a system, a method or a device. In this specification, these implementations, or any other form that the invention may take, may be referred to as processes. In general, the order of the steps of the disclosed processes may be altered within the scope of the invention.

Various terms as used herein are shown below. To the extent a term used in a claim is not defined below, it should be given the broadest definition persons in the pertinent art have given that term as reflected in printed publications and issued patents at the time of filing.

The “shungite stone” also known as natural fullerene stone, black tuff stone or black shell. The shungite stone as used herein is obtained from Russia for purification or treatment of water research. The main component of shungite is carbon C60 and carbon content may reach up to 99%. Other content in shungite also includes SiO₂ (57%), TiO₂ (0.2%), Al₂O₃ (4.0%), FeO (2.5%), MgO (1.2%), K₂O (1.5%), and S (1.2%).

The term, “therapeutically effective amount” as used herein refers to an amount of a shungite treated water effective in producing the desired therapeutic response in a particular patient (subject) suffering from a disease or disorder.

The term, “subject” as used herein refers to an animal, preferably a mammal, and most preferably a human. The term “mammal” used herein refers to warm-blooded vertebrate animals of the class ‘mammalia’, including humans, characterized by a covering of hair on the skin and, in the female, milk-producing mammary glands for nourishing the young, the term mammal includes animals such as cat, dog, rabbit, bear, fox, wolf, monkey, deer, mouse, pig and human.

The terms, “treatment”, “treat” and “therapy” and the like as used herein refer to alleviate, slow the progression, attenuation, prophylaxis or as such treat the existing diseases or condition (e.g. bacterial infection or viral infection). Treatment also includes treating, preventing development of, or alleviating to some extent, one or more of the symptoms of the diseases or condition.

In a general embodiment, the present invention relates to a process for purification or treatment of water using shungite stone.

In another embodiment, the present invention relates to a process of purification or treatment of water comprising the steps of:
(a) crushing shungite stone into 20 mm to 40 mm pieces;
(b) washing the shungite stone pieces using water;
(c) adding the washed shungite stone pieces into the stainless steel tank;
(d) filling the tank with water and stirring the water and the washed shungite stone pieces for 5 to 10 minutes at an interval of 8 hours for 45 to 50 hours; and
(e) filtering the water obtained in step (d) to obtain purified shungite treated water.

In an embodiment of the present invention, the shungite stone is crushed into 20 mm to 35 mm.

In another embodiment of the present invention, the shungite stone is crushed into 20 mm to 25 mm.

In another embodiment of the present invention, the shungite stone is crushed into 25 mm.

In yet another embodiment of the present invention, the shungite stone is crushed into 20 mm.

In yet another embodiment, the shungite stone is crushed into 20 mm, 25 mm, 30 mm, 35 mm and 40 mm in step (a) of the process of present invention. Preferably, the shungite stone is crushed into 20 mm, 25 mm and 30 mm.

In an embodiment of the present invention, the shungite stone is present in an amount ranging from 10 kg to 15 kg for 100 litres of water.

In an embodiment of the present invention, the shungite stone is present in an amount ranging from 12 kg to 13 kg for 100 litres of water.

In an embodiment of the present invention, the shungite stone is present in an amount of 12.5 kg for 100 litres of water.

In an embodiment, the process for purification or treatment of water of the present invention, wherein the duration of stirring and settling of the water and shungite stone is in the range of 40 to 55 hours, preferably in the range of 45 to 50 hours and most preferably in the range of 47 to 48 hours.

In an embodiment of the present invention, the duration of stirring and settling of the water and shungite stone is 48 hours.

In an embodiment, the process for purification or treatment of water of the present invention, wherein the stainless steel tank comprises taps to withdraw the water for filtering.

In an embodiment of the present invention, the stainless steel tank comprises inlet to receive the water and opening to add the shungite stone into the tank. In an embodiment, the stainless steel tank comprises outlet to remove the shungite stone after the completion of the process of preparation of shungite water.

In another aspect, the purified shungite treated water of the present invention possesses various therapeutic effects.

In another aspect, the purified shungite treated water of the present invention complies with the requirement of WHO standards of drinking water.

In another aspect, the purified shungite treated water of the present invention is complies with the toxic substance requirement and organoleptic and physical parameter requirement.

In yet another aspect, the present invention provides a process for purification or treatment of water using shungite, wherein the purified shungite treated water is free of yeast and moulds, Pseudomonas aeruginosa, Escherichia coli, Coliform, Staphylococcus aureus, Faecal Streptococci, Shigella, Salmonella, Vibrio cholera, Vibrio parahaemolyticus, and Sulphite Reducing Anaerobes.

In yet another aspect, the present invention provides a process for purification or treatment of water using shungite, wherein the purified shungite treated water having antibacterial activity, particularly against Pseudomonas aeruginosa, Escherichia coli, Staphylococcus aureus, Faecal Streptococci, Shigella, Salmonella, Vibrio cholera and Vibrio parahaemolyticus.

In an embodiment, there are provided methods for treatment, control or prevention of bacterial infection using the purified shungite treated water according to the invention. In some embodiments, there is provided a method for treatment, control or prevention of bacterial infection in a subject, said method comprising administering to said subject an effective amount of a purified shungite treated water according to the invention.

In another embodiment, the purified shungite treated water according to the present invention is used in treatment, control or prevention of bacterial infection. In some embodiments, the purified shungite treated water according to the invention is used in the manufacture of a medicament for treatment, control or prevention of a bacterial infection.

In an embodiment, there are provided methods for treatment, control or prevention of viral infection using the purified shungite treated water according to the invention. In some embodiments, there is provided a method for treatment, control or prevention of viral infection in a subject, said method comprising administering to said subject an effective amount of a purified shungite treated water according to the invention.

In another embodiment, the present invention relates to use of purified shungite water for treatment, control or prevention of a viral infection.

In another embodiment, the purified shungite treated water according to the present invention is used in treatment, control or prevention of viral infection. In some embodiments, the purified shungite treated water according to the invention is used in the manufacture of a medicament for treatment, control or prevention of a viral infection.

In an embodiment, there are provided methods for treatment, control or prevention of inflammatory diseases using the purified shungite treated water according to the invention. In some embodiments, there is provided a method for treatment, control or prevention of inflammatory diseases in a subject, said method comprising administering to said subject an effective amount of a purified shungite treated water according to the invention.

In another embodiment, there is provided a method for treatment, control or prevention of inflammatory diseases in a subject, said method comprising parenteral administration of a purified shungite treated water according to the invention.

In another embodiment, the purified shungite treated water according to the present invention is used in treatment, control or prevention of inflammatory diseases. In some embodiments, the purified shungite treated water according to the invention is used in the manufacture of a medicament for treatment, control or prevention of inflammatory diseases.

In another embodiment, the purified shungite treated water of the present invention is useful in treatment, control or prevention of gastrointestinal diseases such as heartburn, flatulence, gastritis, colitis, and enteritis, diseases of gallbladder, liver and pancreas (including cholecystitis, pancreatitis and cholelithiasis).

In another embodiment, the purified shungite treated water of the present invention is useful in treatment, control or prevention of cardiovascular diseases such as arterial hypertension, atherosclerosis, angina pectoris, vegeto-vascular dystonia, varicose veins, and diabetic angiopathy.

In another embodiment, the purified shungite treated water of the present invention is useful in treatment, control or prevention of inflammatory and allergic skin diseases such as allergic dermatitis, neurodermatitis, psoriasis, eczema, fungal skin diseases, boils, herpes, traumatic skin lesions for burns, cuts, abrasions, bedsores and calluses.

In another embodiment, the purified shungite treated water of the present invention is useful in treatment, control or prevention of diseases of the joints and spine such as osteochondrosis, radiculitis, arthritis and arthrosis.

In another embodiment, there is provided a method for treatment, control or prevention of viral infection in a subject, said method comprising parenteral administration of a purified shungite treated water according to the invention.

In an embodiment, there are provided methods for treatment, control or prevention of cancer using the purified shungite treated water according to the invention. In some embodiments, there is provided a method for treatment, control or prevention of cancer in a subject, said method comprising administering to said subject an effective amount of a purified shungite treated water according to the invention.

In another embodiment, there is provided a method for treatment, control or prevention of cancer in a subject, said method comprising parenteral administration of a purified shungite treated water according to the invention.

The present invention is further explained in the form of following examples. However, it is to be understood that the following examples are merely illustrative and are not to be taken as limitations upon the scope of the invention.

### Example 1: Purification and Treatment of Tap Water with Shungite Stone

The shungite stone (12.5 kg) was crushed into 10 mm to 40 mm pieces and washed using water (5 litres×5 times). The washed shungite stone was transferred to 100 liter stainless steel tank and added raw water i.e., tapwater (100 litres). The mixture was stirred for 10 minutes and allowed to settle down the shungite stone. The stirring was repeated at an interval of 8 hours up to 48 hours. After 48 hours, the water was filtered through filter to obtain the shungite treated water. The purified shungite treated water obtained was tested for different parameters and the results are provided below.

Microbiological Test for Drinking Water as Per IS 14543:2016 Amendment-1

The shungite treated water obtained above was tested for its microbiological standard suitable for drinking water. The study was conducted at Vimta Laboratories, Hyderabad, India. The result of the study is shown in the table below.

TABLE 1
Microbiological test for drinking water

| S. | | | | Require- | |
|---|---|---|---|---|---|
| No | Test Parameters | UOM | Methods | ments | Results |
| 1 | 5.2.7 Yeast & | Per 250 mL | IS: 5403-1999 | Absent | Absent |
| | Moulds | | | | |
| 2 | 5.2.5 | Per 250 mL | IS: 13428: 2005 | Absent | Absent |
| | Pseudomonas | | | | |
| | aeruginosa | | (Annex. D) | | |
| 3 | 5.2.1 Escherichia | Per 250 mL | IS: 15185-2016/ | Absent | Absent |
| | coli | | ISO 19250: 2010 | | |
| 4 | 5.2.2 Coliform | Per 250 mL | IS: 15185-2016/ | Absent | Absent |
| | | | ISO 19250: 2010 | | |
| 5 | 5.2.3 | Per 250 mL | IS: 5887 | Absent | Absent |
| | Staphylococcus | | | | |
| | aureus | | [Part-II]: 1976 | | |
| 6 | 5.2.3 Faecal | Per 250 mL | IS: 15186: | Absent | Absent |
| | Streptococci | | 2002 RA2009 | | |
| 7 | 5.2.8 Shigella | Per 250 mL | IS: 5887 | Absent | Absent |
| | | | [Part-VII]: 1999 | | |
| 8 | 5.2.8 Salmonella | Per 250 mL | IS 15187: 2016/ | Absent | Absent |
| | | | ISO 12950: 2010 | | |
| 9 | 5.2.9 Vibrio | Per 250 mL | IS: 5887 | Absent | Absent |
| | cholera | | [Part-V]: 1976 | | |
| 10 | 5.2.9 Vibrio | Per 250 mL | IS: 5887 | Absent | Absent |
| | parahaemolyticus | | [Part-V]: 1976 | | |
| 11 | 5.2.4 Sulphite | Per 250 mL | IS: 13428: 2005 | Absent | Absent |
| | Reducing | | (Annex. C) | | |
| | Anaerobes | | | | |
| | 5.2.6 Aerobic | | | | |
| | microbial | | | | |
| | count at | | | | |
| 12 | a) at 37° C., | Cfu/mL | IS: 5402: 2012/ | 20 | <1 |
| | per mL, Max. | | ISO: 4833: 2003 | Cfu/mL | |
| 13 | b) at 22° C., | Cfu/mL | IS: 5402: 2012/ | 100 | <1 |
| | per mL, Max. | | ISO: 4833: 2003 | Cfu/mL | |

The above test results clearly demonstrate that the shungite treated water of the present invention surprisingly suitable for drinking and fulfilsIS 14543:2016 requirement for packaged drinking water.

Radio Activity Study

The shungite treated water obtained above was tested for its radioactivity. The study was conducted at Vimta Laboratories, Hyderabad, India. The result of the study is shown in the table below.

### A.

TABLE 2
Organoleptic and Physical Parameters

| S. | Test | | | | Limit of | |
|---|---|---|---|---|---|---|
| No | Parameters | UOM | Method | Requirements | detection | Results |
| 1 | Colour | True | IS: 3025 (PART 4) - | Max 2 | 1.0 | 1.0 |
| | | Colour | 1983 | | | |
| 2 | Odour | — | IS: 3025 PART 5: 1983 | | Agreeable | Agreeable |
| 3 | Turbidity | NTU | IS: 3025 PART 10: 1984 | Max 2 | 0.1 | <0.1 |
| 4 | Dissolved | Mg/L | IS: 3025 PART 16: 1984 | Max 500 | 2.0 | 58.0 |
| | solids at | | | | | |
| | 180° C. | | | | | |
| 5 | Taste | — | IS: 3025 Part 7&8: 1987 | Agreeable (Action | — | Agreeable |
| | | | | Tendency Scale A | | |
| | | | | or B or C) | | |
| 6 | pH at 25° C. | — | IS: 3025 PART 11: 1983 | 6.0-8.5 | | 6.59 |

The results in the table above demonstrate that the shungite treated water fulfils the organoleptic and physical parameter standards of packaged drinking water and drinking water.

### B. General Parameters Concerning Substances Undesirable in Excessive Amounts

The shungite treated water as obtained above was tested for general parameters concerning substances undesirable in excessive amounts and surprisingly found that the shungite treated water fulfils the standard as required.

### C. General Parameters Concerning Toxic Substances

The shungite treated water obtained above was tested for general parameters concerning toxic substance. We found surprisingly that the purified shungite treated water of the present invention clearly fulfils the WHO standards.

### Example 2: Purification and Treatment of Reverse Osmosis Water with Shungite Stone

The shungite stone (12.5 kg) was crushed into 10 mm to 40 mm pieces and washed using water (5 litres×5 times). The washed shungite stone was transferred to 100 liter stainless steel tank and added reverse osmosis water (100 litres). The mixture was stirred for 10 minutes and allowed to settle down the shungite stone. The stirring was repeated at an interval of 8 hours up to 48 hours. After 48 hours, the water was filtered through filter to obtain the shungite treated water known as shungite water.

The purified shungite treated water obtained was tested for different parameters and surprisingly found that the water fulfils WHO requirement.

### Example 3: Purification and Treatment of Reverse Osmosis, UV and Ozonation Processed Water with Shungite Stone

The shungite stone (12.5 kg) was crushed into 10 mm to 40 mm pieces and washed using water (5 litres×5 times). The washed shungite stone was transferred to 100 liter stainless steel tank and added reverse osmosis, UV and ozonation processed water (100 litres). The mixture was stirred for 10 minutes and allowed to settle down the shungite stone. The stirring was repeated at an interval of 8 hours up to 48 hours. After 48 hours, the water was filtered through filter to obtain the purified shungite treated water known as shungite water.

The purified shungite treated water obtained was tested for different parameters and surprisingly found that the water fulfils WHO requirement.

### Example 4: Therapeutic Activity

The purified shungite treated water as obtained above was tested for its therapeutic activity. The antibacterial activity and antiviral activity studies for various strains were conducted at Vimta Laboratories, Hyderabad, India. The result of the studies are shown in the tables below.

TABLE 3
Antibacterial Activity

| | | | | Count | Count | |
|---|---|---|---|---|---|---|
| S. | Test Parameters | | | at | at 2.5 | Results/ |
| No | (Strain) | UOM | Method | 0 hour | hours | Death |
| 1 | AMC (Bacteria) at 37° C. | cfu/mL | IS: 5402:2012/ISO: 4833:2003 | 500000 | 200000 | 300000 |
| 2 | AMC (Bacteria) at 22° C. | cfu/mL | IS: 5402:2012/ISO: 4833:2003 | 550000 | 250000 | 300000 |
| 3 | Pseudomonas aeruginosa | cfu/mL | IS: 13428:2005 (Annex. D) | 995000 | 35000 | 645000 |
| 4 | E.coli | cfu/mL | IS: 15185-2016/ISO 9308-1:2014 | 460000 | 1500 | 458500 |
| 5 | Staphylococcus aureus | cfu/mL | IS: 5887 [Part-11]: 1976 | 2000000 | 1800000 | 200000 |
| 6 | Faecal Streptococci | cfu/mL | IS: 15186:2002 RA2009 | 1000000 | 950000 | 50000 |
| 7 | Shigella | cfu/mL | IS: 5887 [Part-VII]: 1999 | 450000 | 180000 | 270000 |
| 8 | Salmonella | cfu/mL | IS 15187:2016/ISO 19250:2010 | 400000 | 200000 | 200000 |
| 9 | Vibrio cholera | cfu/mL | IS: 5887 [Part-V]: 1976 | 960000 | 20000 | 940000 |
| 10 | Coliforms | cfu/mL | IS: 15185-2016/1SO 9308-1:2014 | 480000 | 1700 | 478300 |
| 11 | Vibrio parahaemoiyticus | cfu/mL | IS: 5887 [Part-V]: 1976 | 1900000 | 10000 | 1890000 |

TABLE 4
Antiviral Activity

| S. | | | | | Count at | Results/ |
|---|---|---|---|---|---|---|
| No | Test Parameters | UOM | Method | Count at 0 hour | 2.5 hours | Death |
| 1 | MS2 Phage | cfu/mL | USEPA 1602:2001 | 731820 | 1800 | 730020 |

The test results in the above tables clearly demonstrate that the purified shungite treated water of the present invention surprisingly kills the microorganisms and thereby possess antibacterial and antiviral activities.

A skilled artisan will appreciate that the quantity and type of each ingredient can be used in different combinations or singly. All such variations and combinations would be falling within the scope of present disclosure

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

We claim:

1. A process for purification or treatment of water comprising the steps of:
(a) crushing shungite stone into 20 mm to 40 mm pieces;
(b) washing the shungite stone pieces using water;
(c) adding the washed shungite stone pieces into the stainless steel tank;
(d) filling the tank with water and stirring the water and the washed shungite stone pieces for 5 to 10 minutes at an interval of 8 hours for 45 to 50 hours; and
(e) filtering the water obtained in step (d) to obtain purified shungite treated water.

2. The process for purification or treatment of water as claimed in claim 1, wherein the purified shungite treated water is antibacterial water or antiviral water.

3. The process for purification or treatment of water as claimed in claim 1, wherein the water for purification or treatment is tap water, reverse osmosis water or reverse osmosis, UV and ozonation processed water.

4. The process for purification or treatment of water as claimed in claim 1, wherein the shungite stone is in an amount ranging from 10 kg to 15 kg for 100 litres of water.

5. The process for purification or treatment of water as claimed in claim 1, wherein the shungite stone is in an amount of 12.5 kg for 100 litres of water.

6. The process for purification or treatment of water as claimed in claim 1, wherein in step (d) the water and shungite stone pieces were stirred for 5 minutes at an interval of 8 hours for 48 hours.

7. The process for purification or treatment of water as claimed in claim 1, wherein the purified shungite treated water is free of yeast and moulds, Pseudomonas aeruginosa, Escherichia coli, Coliform, Staphylococcus aureus, Faecal Streptococci, Shigella, Salmonella, Vibrio cholera, Vibrio parahaemolyticus, and Sulphite Reducing Anaerobes.

8. The process for purification or treatment of water as claimed in claim 1, wherein the purified shungite treated water possess antibacterial activity against Pseudomonas aeruginosa, Escherichia coli, Staphylococcus aureus, Faecal Streptococci, Shigella, Salmonella, Vibrio cholera and Vibrio parahaemolyticus.

9. The process for purification or treatment of water as claimed in claim 1, wherein the purified shungite treated water is useful in the treatment, control or prevention of bacterial infection, viral infection, anti-inflammatory diseases, cancer, cardiovascular diseases or gastrointestinal diseases.

10. The process for purification or treatment of water as claimed in claim 1, wherein the purified shungite treated water fulfills the WHO requirement of drinking water.

11. A method of treatment control or prevention of bacterial infection or viral infection, the method comprising administering therapeutically effective amount of the shungite treated water obtained in claim 1 to a subject in need thereof.

12. A method of treatment control or prevention of anti-inflammatory diseases, cancer, cardiovascular diseases or gastrointestinal diseases, the method comprising administering therapeutically effective amount of the shungite treated water obtained in claim 1 to a subject in need thereof.
